(19) United States
(12) Patent Application Publication
(10) Pub. No.: US 2012/0016261 A1
(43) Pub. Date: Jan. 19, 2012

(54) HOLLOW MICROTUBE STRUCTURE, PRODUCTION METHOD THEREOF AND BIOPSY DEVICE

(75) Inventors: Makoto Ishida, Aichi-ken (JP); Takeshi Kawano, Aichi-ken (JP); Takahiro Kawashima, Aichi-ken (JP); Kuniharu Takei, Aichi-ken (JP)

(73) Assignee: NAT. UNIV. CORP. TOYOHASHI UNIV. OF TECHNOLOGY, Aichi-ken (JP)

(21) Appl. No.: 13/257,721

(22) PCT Filed: Mar. 19, 2010
(86) PCT No.: PCT/JP2010/054893

§ 371 Date: Sep. 20, 2011

(30) Foreign Application Priority Data
Mar. 20, 2009 (JP) 2009-069216

(51) Int. Cl. A61B 10/02; H01L 21/28; H01L 23/48

(52) U.S. Cl. 600/565; 257/774; 438/667; 257/E23.011; 257/E21.158

(57) ABSTRACT

A hollow microtube structure capable of being used as a minimally invasive electrode, a production method thereof, and a biopsy device using the hollow microtube structure. The hollow microtube structure includes a semiconductor substrate and at least one hollow tube formed on a surface of the semiconductor substrate. The hollow tube includes a metal coating film layer on the inner surface and an electrically insulating coating film layer on the outer surface. The semiconductor substrate includes a through hole communicated with an interior of a hollow tube at a location where the hollow tube is formed. The production method includes an etching, a sacrificial layer forming, a metal coating film layer forming, an electrically insulating coating film layer forming, a tip portion removing, and a piercing. The biopsy device can be provided on a substrate side of the hollow microtube structure with at least one of an electric signal transmitter, an optical signal generator, a chemical fluid injector, an electrical measuring device, a chemical measuring device, and an optical measuring device.

### TECHNICAL FIELD

The present invention relates to a hollow microtube structure, a production method thereof and a biopsy device.

### BACKGROUND ART

In recent years, technology for producing microstructures called micro electromechanical systems (MEMS) has tended to be applied in medical field. For instance, NPL 1 and NPL 2 disclose techniques for producing drug delivery devices and signal measuring devices using techniques for producing MEMS. In addition, NPL 3 discloses a technique for forming a drug delivery tube structure and a signal measuring probe electrode on the same substrate.

Moreover, as an example of techniques for producing a signal measuring probe electrode, PTL 1 discloses a technique for forming an acicular protrusion by vapor-liquid-solid (VLS) growth method. As an example of techniques for producing a drug delivery tube, PTL 2 discloses a technique for etching the abovementioned probe electrode as a sacrificial layer with a coating film layer around the electrode left intact.

### Citation List

### Patent Literature

[PTL 1] Japanese Unexamined Patent Publication No. 2000-333,921

[PTL 2] Japanese Unexamined Patent Publication No. 2007-216,325

### Non Patent Literature

[NPL 1] L. Lin et al. IEEE Journal of Microelectromechanical Systems, Vol. 8, No. 1, pp.78-84 (1999)

[NPL 2] L. R. Hochberg et al. Nature, Vol. 442, pp.164-171 (2006)

[NPL 3] K. Takei et al. Journal of Micromechanics and

Microengineering, Vol. 18, 035033 (2008)

### SUMMARY OF INVENTION

### Technical Problem

For application of MEMS in medical field, especially in neurophysiology, micro probe electrodes and hollow tubes are in demand in view of minimal invasiveness, and it is expected to perform nerve potential measurement and drug delivery using the micro probe electrodes and hollow tubes. Besides, for local neuron analysis, it is required to be capable of performing electrical measurement, chemical measurement, optical measurement and so on simultaneously at the same spot.

However, when a probe electrode and a hollow tube are separately produced, it is difficult to make the electrode and the tube simultaneously contact or invade a local region (e.g., a small region on the order of cells), and a problem is that giving electrical, chemical or optical stimulation and measuring response to the stimulation cannot be performed precisely. Even if the electrode and the tube are formed on the same substrate, leaving intact silicon crystal constituting the probe electrode and removing crystal for forming a tube structure cannot be performed in the vicinity of each other, and as a result the electrode and the tube are located at a distance from each other. Such a structure in which a probe electrode and a tube are located at a distance from each other also has a problem that it is difficult to make the electrode and the tube contact or invade a local region simultaneously.

Besides, in order to realize minimal invasiveness in electric measurement, a tip portion of an electrode is demanded to have a diameter of several micrometers, but in this case, surface area of the electrode tip portion becomes small and electric impedance in biological fluid becomes high. This causes signal attenuation within the probe electrode and when a very small cell signal is to be measured, measurement becomes difficult due to attenuation of nerve potential.

Moreover, when electrodes and tubes function individually with respect to each other and a tube is used to give stimulation by injecting or extracting chemical fluid or the like, an electrode different from the tube is forced to be used in order to measure response to the stimulation. In this way, the number of tubes and that of electrodes respectively change in accordance with what to be measured. As a result, a number of electrodes and tubes have to be prepared in order to meet a variety of measurement requirements.

Therefore, it is an object of the present invention to provide a hollow microtube structure capable of being used as a minimally invasive electrode in order to be capable of giving electric, chemical and/or optical stimulation to a local area and at the same time measuring response to the stimulation, a production method thereof, and a biopsy device using the hollow microtube structure.

### Solution to Problem

The present invention has been made to attain the above object. A hollow microtube structure according to an aspect of the present invention comprises a semiconductor substrate; at least one hollow tube having a hollow portion extending rectilinearly in a perpendicular direction to a front surface of the semiconductor substrate, and provided in a hollow cylindrical shape on a micro scale on the front surface; a metal coating film layer constituting an inner surface of the hollow tube; an electrically insulating coating film layer constituting an outer surface of the hollow tube; and a through hole extending in alignment, while communicated, with the hollow portion of the hollow tube and reaching a back surface of the semiconductor substrate.

With this constitution, since a metal coating film layer forms an inner surface of a hollow tube, the hollow tube can serve as an electrode by filling a hollow portion with normal saline solution, for instance. Besides, since the metal coating film layer is surrounded by an electrically insulating coating film layer, the metal coating film layer at least except an edge of a tip portion is entirely electrically insulated, and when an electric signal is transmitted through the metal coating film layer, electric connection with surroundings of the hollow tube is interrupted and electrical stimulation to non-target areas can be eliminated. Moreover, since a through hole communicated with an interior of the hollow tube is provided in a substrate, from a tip portion of the hollow tube a neuron, for instance, can be given chemical stimulation by injecting chemical fluid or the like from a back side of the substrate, and optical stimulation by radiating light similarly.

The structure according to the above aspect of the invention can be constituted such that the semiconductor substrate is a silicon substrate, the hollow tube has a two-layer structure comprising the metal coating film layer and the electrically insulating coating film layer, and a layered body comprising a metal coating film layer and an electrically insulating coating film layer, continuing from the respective layers constituting the hollow tube, is laid on the front surface of the silicon substrate with the electrically insulating coating film layer disposed on an outer side.

With this constitution, while a hollow tube having the abovementioned effects is provided on a front surface of a silicon substrate, an electrically conductive portion connected to a metal film within the hollow tube can be provided on the front surface of the substrate. This allows a change in electrical potential in a living organism to be transmitted. Moreover, a semiconductor chip which measures and analyzes the change in potential can be constituted by forming a semiconductor integrated circuit on the substrate.

Moreover, the structure according to the respective aspects of the invention can be constituted such that the at least one hollow tube is a plurality of hollow tubes formed in an array on the front surface of the substrate. With this constitution, very small hollow tubes on a nanoscale can be formed on the same substrate in the vicinity of each other. As a result, it becomes possible to give electrical stimulation, chemical stimulation, and/or optical stimulation to cell fibers, for instance, and at the same time with this, measure a change in electrical potential of a target region in response to the stimulation.

On the other hand, a method for producing a hollow microtube structure according to another aspect of the present invention comprises an etching step of etching both a front surface and a back surface of a semiconductor substrate; a sacrificial layer forming step of forming a cylindrical body in an etched area of a front side of the semiconductor substrate; a metal coating film layer forming step of forming a metal coating film layer around the cylindrical body; an electrically insulating coating film layer forming step of forming, around the metal coating film layer, an electrically insulating coating film layer comprising a different material from the cylindrical body; a tip portion removing step of removing the electrically insulating coating film layer and the metal coating film layer of a tip portion of the cylindrical body, thereby exposing the tip portion of the cylindrical body; and a piercing step of removing the cylindrical body and piercing the semiconductor substrate.

In this production method, a cylindrical body serves as a sacrificial layer. When a metal coating film layer and an electrically insulating coating film layer are laid on the cylindrical body, the cylindrical body constitutes a core of a rod-shaped body, and a hollow tube is formed by removing the core. This sacrificial layer also serves as a basic die for forming the metal coating film layer and the electrically insulating coating film layer in a layered structure, and the length and diameter of the sacrificial layer determine the length and inner diameter of a hollow tube to be formed. Moreover, upon removing the cylindrical body and piercing the substrate, inner space of the hollow tube formed on a front side of the substrate and a back side of the substrate can be communicated with each other.

The method of the abovementioned aspect of the present invention can be constituted such that the etching step includes a step of forming a film on both the front surface and the back surface of the semiconductor substrate, a step of removing part of the films, and a step of etching film-removed areas of the semiconductor substrate; and the piecing step is a step of removing the cylindrical body and piecing the semiconductor substrate until reaching one of the etched region of the semiconductor substrate.

With this constitution, only an appropriate area of the film on the back side of the substrate is removed, so only a desired position of the substrate can be etched away. Besides, a hollow tube to be formed on a front side and a region to be etched (to be pierced) on a back side can be aligned in a straight line. Since the region of the substrate to be etched away from the back side of the substrate is made larger than inner space of the hollow tube in order to ensure piercing in the piercing step, an appropriate amount of space is formed on the backside of the substrate. This space can not only serve as a fluid reservoir in injecting chemical fluid or the like but also store a connector for connection to a variety of devices.

The method according to the respective aspects of the prevent invention can be constituted such that the metal coating film layer forming step is a step of forming a metal coating film layer on the front surface of the substrate simultaneously with forming the metal coating film layer around the cylindrical body, and the electrically insulating coating film layer forming step is a step of forming an electrically insulating coating film layer on the metal coating film layer disposed on the front surface of the substrate simultaneously with forming the electrically insulating coating film layer around the metal coating film layer disposed around the cylindrical body.

With this constitution, an electrically conductive portion which is electrically connected to the inner surface of the hollow tube can be formed on the front surface on which the hollow tube is formed. This allows a semiconductor integrated circuit to be formed on the substrate. When this kind of integrated circuit is formed, it is also possible to form a semiconductor chip which measures and analyzes a change in potential in a living organism.

Next, a biopsy device using the hollow microtube structure according to another aspect of the present invention is a biopsy device using the hollow microtube structure according to any one of claims 1 to 3 and comprising the hollow microtube structure; chemical fluid injecting means provided continuously to an opening portion of the through hole on a back side of the substrate and supplying chemical fluid to an interior of the hollow portion of one of the at least one hollow tube; and electric signal transmitting means and electrical measuring means each electrically connected to the one or another of the at least one hollow tube.

With this constitution, it is possible to inject chemical fluid, for example, to neurons by using one hollow tube, and measure response of the neurons by electrical measuring means which is electrically connected to another hollow tube. In this case, states of a living organism can be checked by response of neurons or the like to a certain chemical fluid. It is also possible to give electrical stimulation, for example, to neurons by using one hollow tube from electric signal transmitting means electrically connected to the hollow tube, and measure response of the neurons by electrical measuring means which is electrically connected to another hollow tube.

Furthermore, when normal saline solution, for instance, is injected by the chemical fluid injecting means into the hollow tubes which are respectively electrically connected to the electric signal transmitting means and the electrical measuring means, the inner space of the hollow tubes are filled with the normal saline solution. As a result, electric signals can be transmitted and received while keeping signal attenuation rate low. In addition to the above, in a case of using a hollow microtube structure having hollow tubes in an array, a plurality of hollow tubes can be formed in the immediate vicinity of each other. Therefore, it is also possible to give electrical stimulation (stimulation given by electric signal transmitting means) or chemical stimulation (stimulation given by chemical fluid injecting means), for example, to the same neuron and at the same time electrically measure response of the neuron to the stimulation, for example, in the form of a change in electrical potential.

A biopsy device according to another aspect of the present invention can be a biopsy device using the hollow microtube structure according to any one of claims 1 to 3 and comprising the hollow microtube structure; optical signal generating means for emitting a light source to enter from an opening portion of the through hole on a back side of the substrate and pass through one of the at least one hollow tube; and optical measuring means for receiving reflected light of the light source which has reached the back side of the substrate through an interior of the hollow portion of another of the at least one hollow tube.

With this constitution, light can be radiated to an inside of a living organism (for example, neurons) by using one hollow tube and light reflected by the inside of the living organism can be received by using another hollow tube. Therefore, it is possible to conduct optical analysis of part of a living organism by using reflected light. This reflected light can conduct not only a test of measuring optical response to optical stimulation by light radiation, but also a test of checking colors of cells and their surroundings.

A biopsy device according to still another aspect of the present invention can be a biopsy device using the hollow microtube structure according to any one of claims 1 to 3 and comprising the hollow microtube structure; optical signal generating means for emitting a light source to enter from an opening portion of the through hole on a back side of the substrate and pass through one of the at least one hollow tube; and electrical measuring means electrically connected to the one or another of the at least one hollow tube.

With this constitution, it is possible to radiate light, for example, to retinal cells by using one hollow tube and electrically measure response of the retinal cells by using another hollow tube. This light radiation is to give optical stimulation to the cells and response to the light can be measured in the form of a change in electrical potential.

The biopsy device according to claim 8 or 9 can also be constituted such that the biopsy device further comprises chemical fluid injecting means provided continuously to the opening portion of the through hole on the back side of the substrate and supplying chemical fluid through an interior of the one or another of the at least one hollow tube.

With this constitution, chemical stimulation by injecting chemical fluid can be given at the same time as giving optical stimulation by light radiation. It is also possible to decrease electric signal attenuation by supplying, for example, normal saline solution as chemical fluid and filling an interior of a hollow tube with the normal saline solution, and electrically connecting the electrical measuring means to the same hollow tube.

The biopsy device according to any one of claims 7 to 10 can also be constituted such that the biopsy device further comprises fluid extracting means provided continuously to the opening portion of the through hole on the back side of the substrate and extracting, by suction, body fluid or chemical fluid by way of an interior of the one or another of the at least one hollow tube; and chemical measuring means provided continuously to or on a way to the fluid extracting means.

With this constitution, when electrical stimulation (stimulation given by electric signal transmitting means), chemical stimulation (stimulation given by chemical fluid injecting means) or optical stimulation (stimulation given by optical signal generating means) is given, for instance, to neurons, a change in body fluid around the neurons can be chemically measured and, when the chemical fluid once injected is extracted by suction, a change in the chemical fluid can be chemically measured. It should be noted that chemical analysis can be done by using a reactant which is different with a target (body fluid or a variety of chemical fluids) to be extracted by suction by the fluid extracting means.

### Advantageous Effects of Invention

The hollow microtube structure according to the present invention can give a local area electrical, chemical and/or optical stimulation in accordance with mode of use and measure response to the stimulation. Furthermore, these measurements can be done simultaneously at the same spot. Moreover, since a micro hollow tube can serve as an electrode, minimally invasive stimulation and measurement can be performed.

The method for producing a hollow microtube structure according to the present invention can efficiently produce a hollow microtube structure having at least one hollow tube on a semiconductor substrate.

The biopsy device according to the present invention can measure response to electric, chemical and/or optical stimulation in a very micro local region, and perform a biopsy with the measurement results. Measurement results can be regarded as biopsy results by specifically limiting what to be inspected, that is to say, by considering that the measuring means for measuring the change is, for example, means capable of measuring a change in potential in a case of the electrical measuring means, means capable of measuring wavelength or the like of received light in a case of the optical measuring means, or means capable of measuring the presence and amount of one or more certain components in a case of the chemical measuring means.

### BRIEF DESCRIPTION OF DRAWINGS

FIG. 1 is cross sectional views for explaining part of a method for producing a hollow microtube structure.

FIG. 2 is cross sectional views for explaining the following part of the method for producing a hollow microtube structure.

FIG. 3 is an explanatory schematic view of a hollow microtube structure according to an embodiment.

FIG. 4 is an explanatory schematic view of a biopsy device according to an embodiment.

FIG. 5 is a model diagram of experimental equipment used in an experiment of measuring electrical potential using a hollow tube.

FIG. 6 is a graph showing evaluation results of output/input signal ratio in measuring potential using a hollow tube.

FIG. 7(a) is a diagram showing a hollow tube electrode model along with its equivalent circuit and FIG. 7(b) is a diagram showing a probe electrode model along with its equivalent circuit.

FIG. 8 is a model diagram of experimental equipment used in an experiment of injecting and extracting fluid through a hollow tube.

FIG. 9 shows microscopic images of fluid injection and extraction using a hollow tube.

FIG. 10 shows microscopic images of light transmission through a hollow tube.

FIG. 11 shows microscopic images of micro object fixation using a hollow tube.

### DESCRIPTION OF EMBODIMENTS

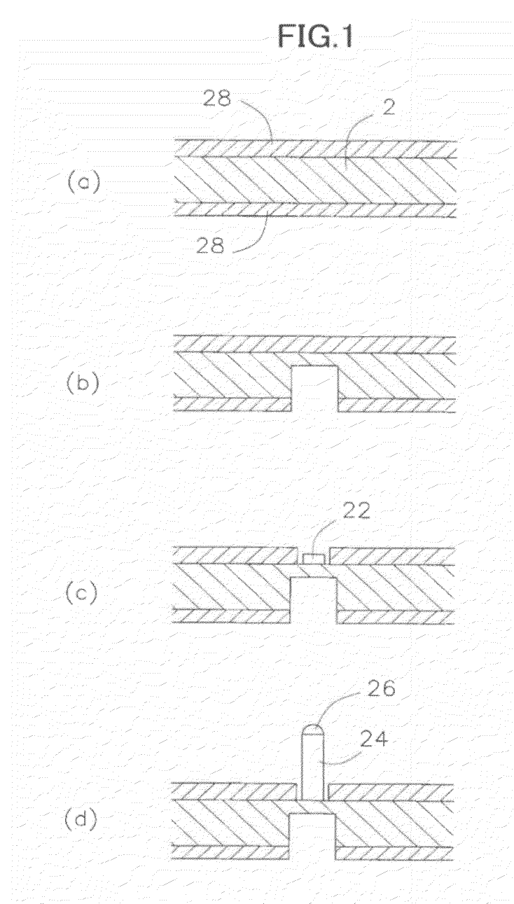

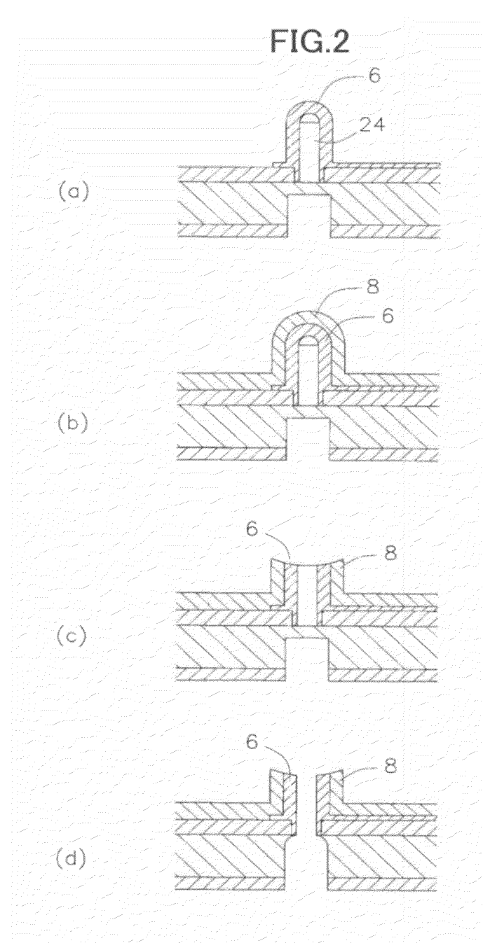

Hereinafter, embodiments of the present invention will be described with reference to the drawings. For convenience, first, an embodiment of a method for producing a hollow microtube structure will be explained, and then embodiments of a hollow microtube structure and a biopsy device will be explained. FIGS. 1 and 2 are cross sectional views showing a method for producing a hollow microtube structure according to the present invention. FIG. 1 show sequences from a preliminary step to a step of forming a cylindrical body, and FIG. 2 show sequences from a step of forming a metal coating film layer to a final step.

Performed as a preliminary step (an etching step) are a step of forming a film 28 on each surface of a semiconductor substrate 2 (FIG. 1(a)) and a main part of the etching step of partially removing the film 28 formed on the back surface of the semiconductor substrate 2 and etching the film-removed area of the substrate 2 (FIG. 1(b)). When a silicon (Si) substrate is used as the semiconductor substrate 2 in the step of forming the films 28, the films 28 can be silicon oxide films. Partial removal of the oxide silicon films can be carried out by dry etching or wet etching, that is to say, by using a method of dry etching or wet etching an area exposed from a patterned photoresist mask. In etching the substrate 2 in the film-removed area, it is desirable to form an etched region having an appropriate depth from the back surface of the substrate 2, that is to say, to etch the substrate 2 so as not to reach the front surface and make a hole in the substrate 2. This is to allow a cylindrical body (a probe) 24 to be formed on the front side of the substrate 2 in a later step. It is desirable that the semiconductor substrate 2 is a substrate having crystal orientation such as a gallium arsenide (GaAs) substrate, in addition to the silicon substrate. It should also be noted that the films 28 are not limited to silicon oxide films and can be other electrically insulating films.

Subsequently, a step of forming a cylindrical body (hereinafter referred to as a probe) 24 (a sacrificial layer forming step) is carried out. In this step, the probe 24 is formed on the front side (i.e., an opposite side to the etched region) of the substrate 2. First, part of the film 28 formed on the front surface of the substrate 2 in the preliminary step is removed and a void portion is formed in the film-removed area. Then, a metal film 22 is placed on the front surface of the substrate 2 in the void portion (FIG. 1(c)). In this case, the void portion is formed in a circular shape and the position of the void portion is adjusted so as to be in alignment with the etched region formed on the back side of the substrate 2 in the preliminary step. The metal film 22 is shaped of a circle having a smaller diameter than that of the void portion, and is located at a center of the circular void portion. This metal film 22 serves as a catalyst for forming the probe 24 and comprises gold, platinum, or the like. The size of the metal film 22 determines the diameter of the probe 24. Since outer diameter of the probe 24 is the same as inner diameter of a hollow tube to be produced in a later step, it is necessary to use a metal film 22 shaped of a circle of several micrometers in diameter in order to produce a hollow tube having a predetermined inner diameter.

Next, the probe 24 is formed so as to stand on the substrate 2 (FIG. 1(d)). Formation of the probe 24 is made by VLS growth method; the probe 8 is grown on the substrate 2 placed in a high vacuum chamber by supplying disilane (Si₂H₆) gas or the like. The height (length) of the probe 24 to be grown is controlled by gas supply time, and the diameter of the probe 24 is controlled by the area or the like of the metal film 22. The sequence heretofore is a step of forming a cylindrical body.

Subsequently, a step of forming a metal coating film layer 6 (a metal coating film layer forming step) is performed. The metal coating film layer 6 is formed so as to cover the entire probe 24 and part of the front side of the substrate 2 (FIG. 2(a)). Of the metal coating film layer formed on the front side of the substrate 2, unnecessary part is removed by etching and the substrate 2 or only necessary part for connection to an external device are left intact. In this step, the metal coating film layer 6 is also formed in a gap between the probe 24 and the film 28 at a base portion of the probe 24. That is to say, a circular void portion is formed by removing part of the film 28 formed on the entire front surface of the substrate 2, but the probe 24 formed in the void portion has a smaller diameter than the void portion. Therefore, part of the void portion forms a gap between the film 28 and the probe 24, and in forming the metal coating film layer 6, a surface of the gap is covered (actually, the metal coating film layer 6 invades the entire gap). Formation of the metal coating film layer 6 can be made by deposition method or sputtering. Examples of metal to be employed include not only gold and platinum but also iridium, silver and silver—silver chloride. When a hollow microtube structure is used for measuring cells, it is especially desirable to form the metal coating film layer 6 from the materials listed here. It should be noted that the metal coating film layer 6 on the front side of the substrate 2 is electrically insulated due to presence of the film 28 between the substrate 2 and the metal coating film layer 6. When such a metal coating film layer 6 is not to be formed partially on the front side of the substrate 2, the metal coating film formed on the front surface of the substrate 2 is removed by etching.

Next, a step of forming an electrically insulating coating film layer 8 (an electrically insulating coating film layer forming step) is carried out. The electrically insulating coating film layer 8 is formed so as to cover both the metal coating film layer 6 formed on a surface of the probe 24 and the metal film formed on the front side of the substrate 2 (FIG. 2(b)). Formation of this electrically insulating coating film layer 8 can be made by deposition method. Not only an oxide film or a nitride film but also a resin can be used as the electrically insulating coating film layer 8. The electrically insulating coating film layer 8 is formed so as to cover the entire front side of the substrate 2 and does not have to be removed partially. However, when the electrically insulating coating film layer 8 is to be partially removed, the electrically insulating coating film layer 8 at least in an area where the metal coating film layer 6 formed in the former step is located should not be removed.

Subsequently, a step of exposing a tip portion of the probe 24 (a tip portion removing step) is performed. Removed here are the electrically insulating coating film layer 8 constituting an outer layer, the metal coating film layer 6 constituting an inner layer, and the metal film 26 which served as a catalyst in the VLS growth method (FIG. 2(c)). Method of removal is not limited and can be wet etching or dry etching. For example, the electrically insulating coating film layer 8 can be removed by selective etching or reactive ion etching (RIE) method, and the metal coating film layer 6 and the metal film 26 can be removed not only by dissolving the metal in aqua regia but also by RIE method.

At this time, by etching the metal coating film layer 6 slightly more than the electrically insulating coating film layer 8, a tip portion of a hollow tube obtained as a final product after a final step can be constituted such that the electrically insulating coating film layer 8 projects from the metal coating film layer 6.

Performed as a final step is a step of removing the probe 24 and piecing the substrate 2 (a piercing step). Etching the substrate 2 at the same time as the probe 24 makes an interior of the metal coating film layer 6 hollow to form a hollow tube, and allows inner space of the hollow tube to be communicated with the back side of the substrate 2 (FIG. 2(d)).

When the probe 24 is silicon and the electrically insulating coating film layer 8 is silicon oxide, the probe 24 can be etched by using xenon difluoride (XeF₂) or iodine fluoride (SF₆). When xenon difluoride is used for etching, silicon oxide is hardly removed because an etching rate of silicon oxide to silicon is 1/100,000. When the probe 24 is gallium arsenide, boron trichloride (BCl₃) can be used as etching gas. This also applies to etching of the substrate 2 (including etching in the preliminary step).

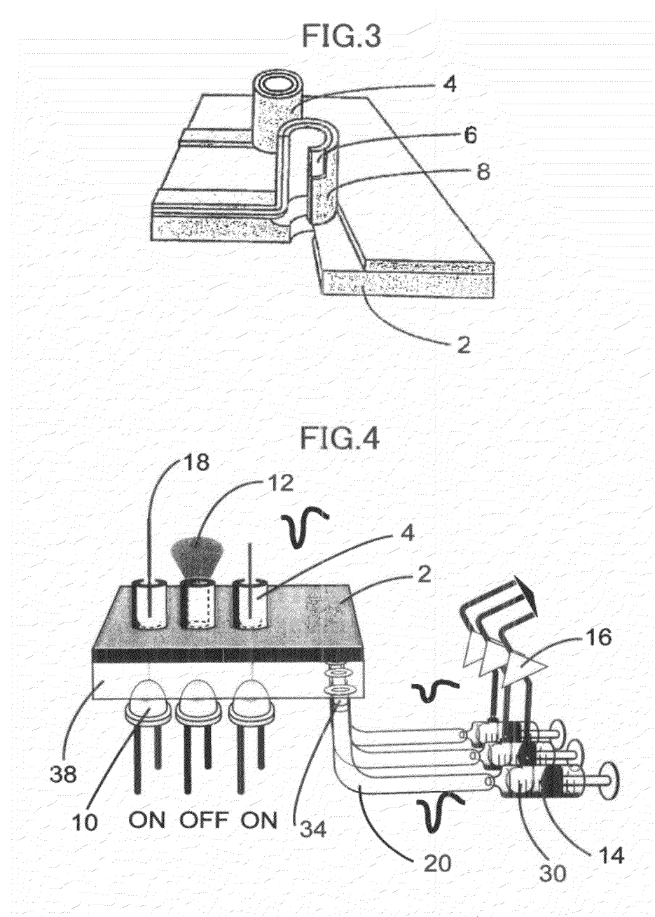

Upon applying treatments according to the aforementioned steps, it becomes possible to obtain a hollow microtube structure in which at least one hollow tube of several micrometers in diameter stands on a semiconductor substrate. Now, an embodiment of the hollow microtube structure will be described. FIG. 3 is a schematic diagram of a hollow microtube structure formed by the abovementioned production method using a silicon substrate.

As shown in FIG. 3, the structure of the present embodiment has hollow tubes 4 standing on a front surface of a substrate 2, and each of the hollow tubes 4 has a metal coating film layer 6 as an inner layer. The metal coating film layer 6 is continuous to a metal coating film layer on a front side of the substrate 2 and is capable of being electrically connected with the front surface of the substrate 2 or an outside of the substrate 2. An electrically insulating coating film layer 8 is formed on an outer side of the metal coating film layer 6 and when the hollow tubes 4 are forced to invade a living organism, the hollow tubes 4 are electrically disconnected from the living organism. It should be noted that the inner diameter of the hollow tubes 4 can be controlled in a production stage, and minimal invasiveness can be attained by forming hollow tubes 4 having an outer diameter of less than 10 μm by controlling the inner diameter in the range of 2 to 7 μm.

Furthermore, an interior of the hollow portion of each of the hollow tubes 4 is communicated with a back side of the substrate 2 by way of an etched region of the substrate 2, inner space of each of the hollow tubes 4 on the front side, which was isolated from the back side due to presence of a main body of the substrate 2, is made continuous to space on the back side of the substrate 2. Moreover, the metal coating film layer 6 constituting an inner layer of each of the hollow tubes 4 is formed even in the neighborhood of the etched region of the substrate 2 and as a result, electrical connection with each of the hollow tubes 4 is also possible on the back side of the substrate 2.

One of the hollow tubes 4 having the abovementioned constitution can serve as a high-performance electrode in spite of very small diameter by filling the hollow tube 4 with normal saline solution. Purpose of filling the hollow tube 4 with normal saline solution is to make the entire hollow tube 4 electrically conductive, and electrical connection between electrical measuring means and a tip portion of the hollow tube is ensured by making normal saline solution present not only inside the hollow tube 4 but also in all the way to the electrical measuring means. With this constitution, reduction of impedance (about 1/10) is achieved owing to resistance value (14.7 ohm cm) of normal saline solution. That is to say, it is possible to obtain an electrode with which a signal does not attenuate even in measuring nerve potential. Accordingly, a change in potential in giving some stimulation to neurons can be measured with this electrode. In this case, a potential measuring signal can be acquired not only from the metal film on the front side but also from the back side of the substrate 1.

Besides, because space inside the hollow tubes 4 is communicated with the back side of the substrate 2, light can be radiated from a tip portion of one of the hollow tubes 4 by placing a light source on the back side of the substrate 2. In this case, upon making the electrically insulating coating film layer 6 an optically shielding coating film layer, optical stimulation other than radiated light can be prevented from leaking into a living organism. Since light can reach a target even when a hollow tube 4 is filled with normal saline solution, it is possible to simultaneously give optical stimulation and measure a response to the stimulation by using a single hollow tube 4.

Moreover, it is possible to measure a change in potential while giving chemical stimulation to a neuron, by filling a hollow tube with a chemically stimulating electrolyte instead of normal saline solution and discharging the electrolyte from a tip portion of the hollow tube. In this case, too, it is possible to give chemical stimulation and measure a response to the stimulation by using a single hollow tube.

Upon simultaneously forming a plurality of hollow tubes 4 as shown in the drawing, the hollow microtube structure can be a structure having hollow tubes arranged in an array on the front side of the substrate 2. Because of being formed on the same substrate, a plurality of electrodes or tubes can be located in the vicinity of each other. When used as an electrode, a hollow tube 4 is filled with normal saline solution, and when used as a hollow body, a hollow tube 4 can be used for taking optical or chemical records or giving optical or chemical stimulation by using inner space.

Specifically, when four hollow tubes 4 are arranged in an array and one of the tubes 4 is used as an electrode for measuring electric potential and the other three are respectively used for light radiation, chemical fluid supply and electrical stimulation, upon simultaneously inserting the respective hollow tubes 4 into a living organism and placing their tip portions in the same local area, it is possible to give optical, chemical and electrical stimulations sequentially or simultaneously and measure response to these stimulations sequentially.

Similarly, when one hollow tube 4 is used as an electrode for measuring electrical potential and the other three are used as hollow bodies for radiating light sources having different wavelengths (e. g., 470 nm, 525 nm, 595 nm), it is possible to measure response to stimulations of different color lights (blue, green, red). This is effective, for example, in inspecting color reaction in neurons constituting the retina.

Upon combining these, it becomes possible to simultaneously give a variety of stimulations and measure responses in a local area and thus, a precise sensory test can be conducted on certain cells (e. g., neurons constituting the retina) in a living organism.

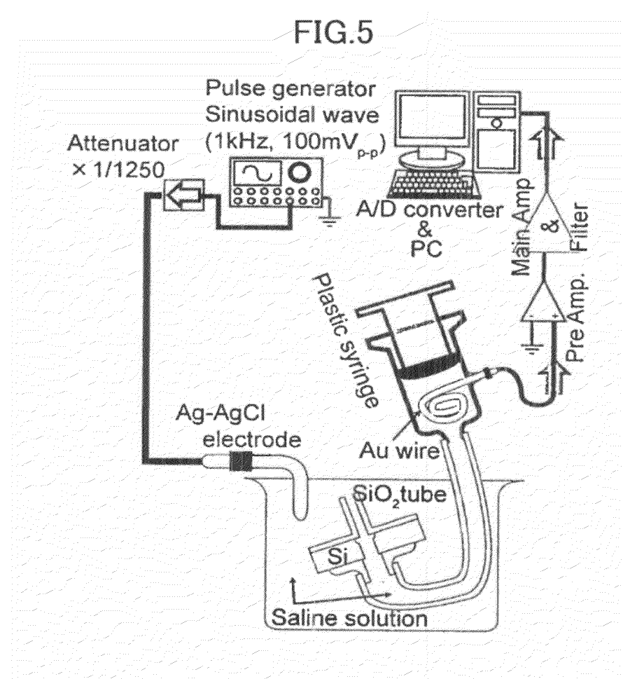

Now, an embodiment of a biopsy device will be described. As shown in FIG. 4, a microchannel-equipped resin 38 is deposited on a back side of a substrate 2 so that respective hollow tubes 4 can secure passages for supplying or suctioning fluid such as normal saline solution and chemical fluid is secured for each hollow tube 4 and a light passage for light radiation.

For permitting fluid supply or suction, this device is provided with connectors 34 for connection to opening portions of the passages of the microchannel-equipped resin 38 so as to be capable of being respectively connected to syringes 14 by way of flexible tubes 20. Besides, for permitting light radiation, the device is constituted such that a light source can be placed at an opening portion of a microhole formed rectilinearly in the microchannel-equipped resin 38. It should be noted that light radiation timing and radiation time control can be facilitated by using a light-emitting diode or a laser diode as a light source.

For electrical potential measurement, normal saline solution is supplied to a hollow tube 4 by one of the syringes 14 and electric connection from the syringe 14 is made by metal wire. In addition to that, as mentioned above, electric potential can be acquired from a metal coating film layer 6 formed on a front side of the substrate 2. When a change in potential is measured from the syringe 14, a desired potential can be amplified and measured by electrically connecting the syringe 14 and an electrical measuring device by way of an amp filter 16. When the metal coating film layer 6 of the substrate 2 is used, a filter circuit can be embedded in the substrate 2.

Conversely, when an electric signal is transmitted to a tip portion of a hollow tube 4, the electric signal can be transmitted from a syringe 14 with the hollow tube 4 supplied with normal saline solution similarly to the above, or the electric signal can be transmitted by way of the metal coating film layer 6 on the front side of the substrate 2.

It should be noted that since fluid present in the vicinity of a tip portion of a hollow tube 4 can be suctioned by using a syringe 14, chemical measurement can be made by suctioning intermittently or continuously and conducting chemical analysis of obtained fluid. Besides, optical measurement of a local region can be made by radiating a light source through a certain hollow tube 4 and observing light such as fluorescence through the same or another hollow tube. Furthermore, when stimulation and measurement are conducted about a micro portion, the micro portion can be fixed at a tip portion of a hollow tube 4 by suction using a syringe 14 and a target portion to be inspected can be inspected without fail.

As mentioned above, upon using the hollow microtube structure, it is possible to locate and operate electric signal transmitting means (an electric signal transmitter), optical signal generating means (an optical signal generator), chemical fluid injecting means (a chemical fluid injector), electrical measuring means (an electrical measuring device), optical measuring means (an optical measuring device), and chemical measuring means (a chemical measuring device). Furthermore, when a syringe is used as chemical fluid injecting means (a chemical fluid injector), the syringe can also serve as fluid extracting means (a fluid extractor) for extracting chemical fluid or body fluid by suction.

Accordingly, when a chemical fluid injector, an electric signal transmitter and an electrical measuring device are provided on a substrate side of a hollow microtube structure, it is possible not only to measure electric response of neurons or the like to injected chemical fluid but also to measure response to electrical stimulation.

When an optical signal generator and an optical measuring device are provided, optical response to optical stimulation can also be measured. Furthermore, electric response to optical stimulation can be measured by providing an optical signal generator and an electrical measuring device. If a chemical fluid injector is provided in addition to these, response to a combination of optical stimulation and stimulation by chemical fluid can be measured.

Especially when a biopsy device is constituted so as to be equipped with an optical signal generator and an electrical measuring device, the device can conduct an optical reaction test of neurons constituting the retina. In this case, response to light's three primary colors, i. e., red, green, blue can be examined by preparing a plurality of optical signal generators and having the light sources emit lights having different wavelengths. Since signals indicating a change in potential obtained through hollow tubes 4 in this test are transmitted without attenuation, precise measurement results can be obtained.

Upon providing a fluid extractor in each of the above devices, it becomes possible to extract chemical fluid which was temporarily placed in a living organism or body fluid or the like changed by the above stimulation. A further biopsy can be performed by analyzing the thus obtained fluid by an analyzer or the like.

Though the embodiments of the present invention have been described as above, a variety of modifications are possible within the gist of the present invention. For example, although the microchannel-equipped resin is deposited on the back side of the substrate 2 in one of the above embodiments of the biopsy device, the biopsy device can be constituted such that a syringe 14 is directly connected to an etched region formed on the back side of the substrate 2 or a light source is provided in the vicinity of an etched region. The biopsy device can also be constituted so as to be capable of simultaneously giving optical stimulation and making electrical measurement by supplying normal saline solution to a hollow tube 4 by way of a flexible tube and providing a light source in the etched region corresponding to the hollow tube 4. Moreover, fluid supply can also be made by integrating a micro pump and using a fluid tank. When the biopsy device has such a constitution, the device can be used by being implanted in a living organism as a compact solution supply system.

### EXAMPLES

Hereinafter, specific examples of the present invention will be described. As a preliminary step, a (111)-oriented silicon substrate was prepared, an oxide coating film was formed on each surface of the substrate, a predetermined area of the silicon oxide coating film on a back side was removed by etching with buffered hydrofluoric acid, and the substrate was etched to a portion close to a front surface, thereby forming an etched region. In a step of forming a cylindrical body, the oxide coating film on a front side of the substrate was etched in a circular shape and a circular metal film having a diameter of 2 μm was placed in a center of the etched area and a probe was allowed to grow to a height of 20 μm by VLS growth method. In a metal coating film layer forming step, a gold coating film layer was formed by deposition method. In an electrically insulating coating film layer forming step, a silicon oxide film was formed by deposition method. In a step of exposing a tip portion of the probe, the electrically insulating coating film layer was etched by RIE method and the metal coating film layer was removed by dissolving the layer in aqua regia. In a final step, the probe was removed by etching with xenon difluoride and the substrate was etched so that the space for the probe could be communicated with the etched region formed on the back side of the substrate.

Thus obtained was a hollow microtube structure in which a hollow tube stood on the substrate and inner space of the hollow tube was communicated with the back side of the substrate by way of the etched region. The hollow tube was shaped of a hollow cylinder and had an inner diameter of 2 μm, an outer diameter of 3 μm and a length of 20 μm. A hollow microtube structure constituting a hollow tube array could be obtained by growing cylindrical bodies (probes) at a plurality of spots and forming a plurality of hollow tubes by using these probes as sacrificial layers in a similar procedure to the above.

Then, experiments were carried out in order to confirm whether a variety of measurements are possible or not by the hollow tube structure produced by the specific example.

### Experiment 1

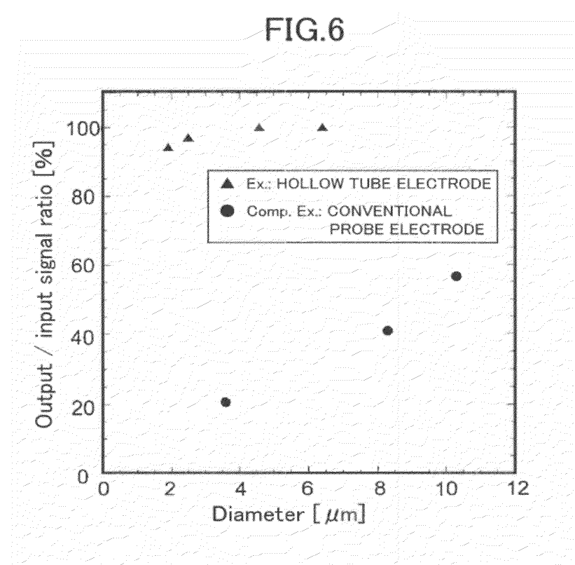

First conducted was evaluation of output/input signal ratio in measuring potential by using a hollow tube electrode produced in the above specific example. Experimental equipment is shown in FIG. 5. An interior of the hollow tube was filled with normal saline solution and gold wire as an electrode was provided inside a cylinder of a syringe. An electric signal (sinusoidal wave of 1 kHz, 100 mVₚ₋ₚ) generated by a pulse generator was attenuated by a resistive attenuator to about 1/1250 (about 80 μVₚ₋ₚ after attenuation), and the attenuated signal was given to the vicinity of a tip portion of the hollow tube. The result is shown in FIG. 6. For reference, FIG. 6 also shows experimental results using the same kind of hollow tubes having different diameters. FIG. 6 also shows experimental results of conventional probe electrodes as comparative examples. These experimental results show that in a case of the conventional probe electrodes, the output/input ratio drastically decreased with a decrease in diameter, but in a case of the hollow tube electrodes of this specific example, the output/input ratio hardly changed in spite of a decrease in outer diameter.

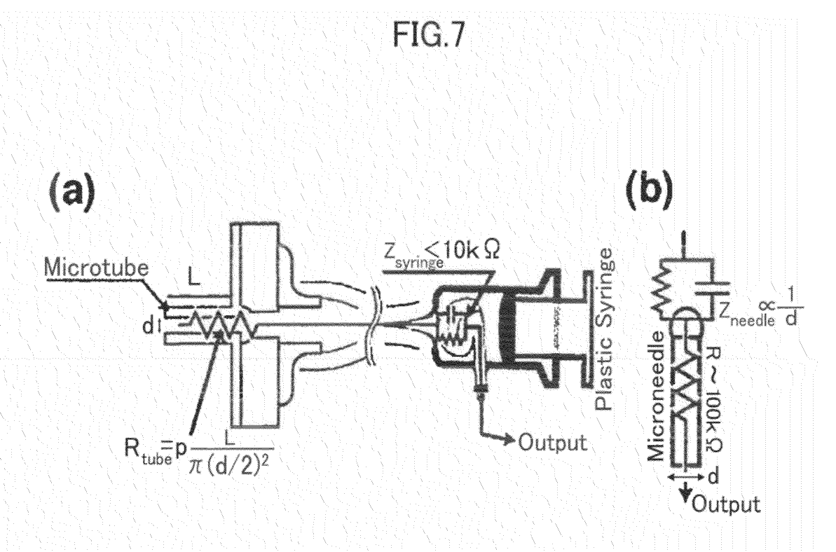

It should be noted that the abovementioned “conventional probe electrodes” are the same as the probes which were produced by crystal growth as sacrificial layers, and their specific constitution is as follows. First, a silicon oxide (SiO₂) film is formed on each side of a (111)-oriented silicon (Si) substrate and catalyst metal (Au) is selectively formed by lift-off process. Then, the substrate is enclosed in a high vacuum atmosphere and silicon (Si₂H₆) gas is supplied for crystal growth by gas-source molecular beam epitaxy method while the substrate is heated at 500 to 700 deg. C. A probe is formed in an n-type semiconductor region of the silicon substrate and an electrode is bonded to a drain region of the substrate. In the experiment, an attenuated electric signal of about 80 μVₚ₋ₚ was output into normal saline solution as mentioned above and the signal was measured with a tip portion of the probe electrode immersed in the normal saline solution. For reference, the hollow tube electrode model of the example and the conventional probe electrode model are shown in FIGS. 7 together with their equivalent circuits.

### Experiment 2

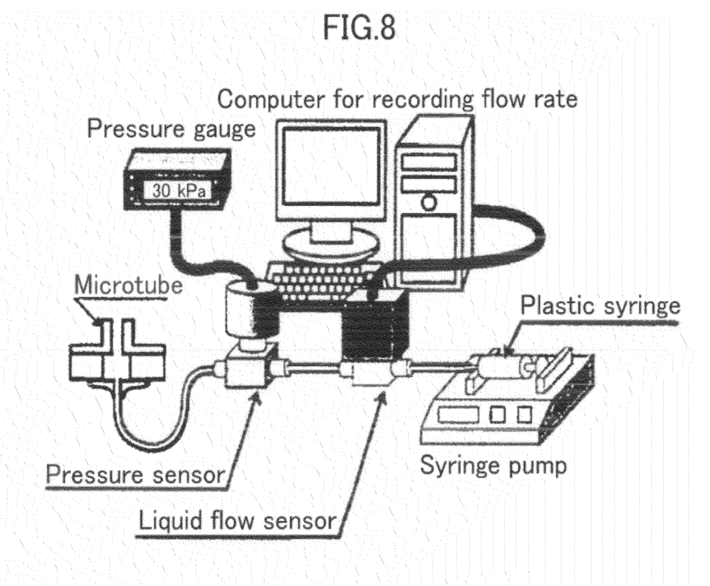

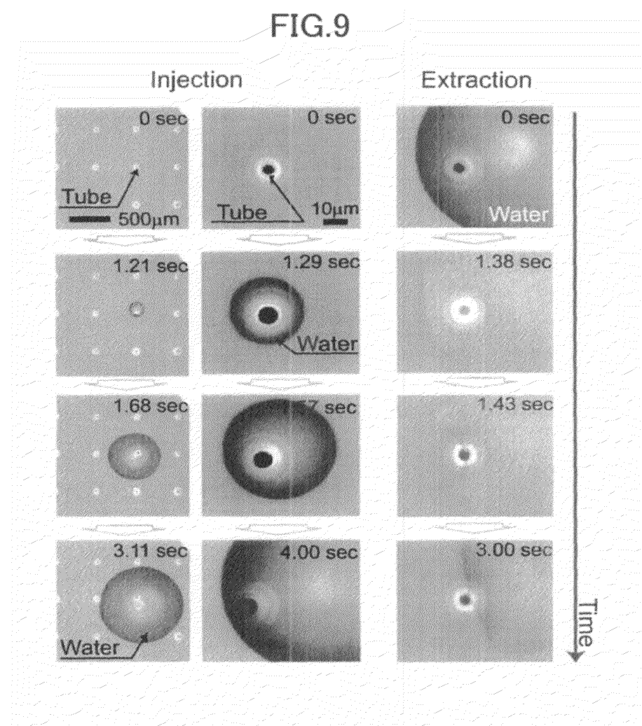

Next, an experiment was conducted on feasibility of fluid injection and extraction. As shown in FIG. 8, experimental equipment was constituted such that pressure was applied on a plastic syringe by a syringe pump (Model 11 Pico Plus produced by Harvard Apparatus) and flow rate was measured by a flow sensor (SLG 1430 produced by Sensirion) capable of sensing extremely small flow rate. Hollow tubes used in this experiment had four kinds of inner diameters, 2.5 μm, 4.1 μm, 4.6 μm and 6.4 μm, and the same length of 22 μm. As a result, all the hollow tubes were capable of injecting fluid. An additional experiment was conducted on feasibility of fluid injection and extraction using a hollow tube having an inner diameter of 2 μm. States in this experiment are shown in FIG. 9. FIG. 9 shows photographs taken from a tip portion side of the hollow tube. As apparent from this figure, it was demonstrated that fluid injection and extraction using hollow tubes are possible.

### Experiment 3

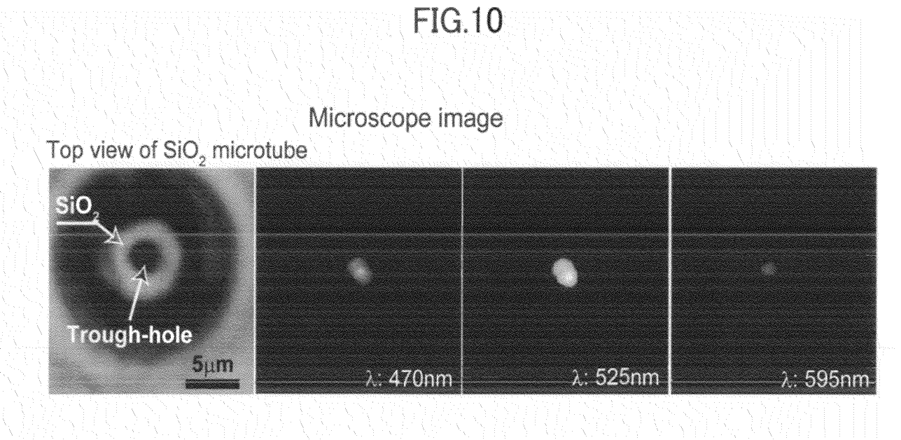

Another experiment was conducted on light transmittance by radiating light from a light-emitting diode on a back side of a substrate 2 and observing light which has reached a tip portion of a hollow tube. The hollow tube had an inner diameter of 2 μm and light to be transmitted was divided by wavelength: 470 nm (blue), 525 nm (green) and 595 nm (red). The results are shown in FIG. 10. These experimental results demonstrated that light with any of the wavelengths passes through a hollow tube.

### Experiment 4

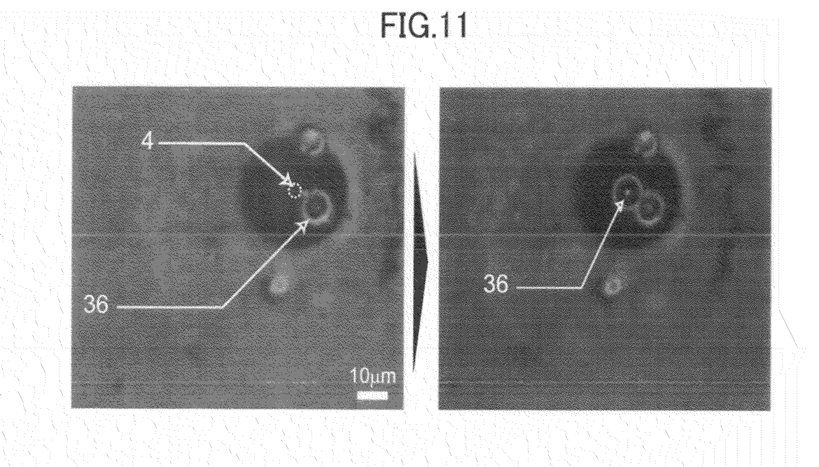

Still another experiment was conducted using a micro bead in order to examine whether a microscopic target such as neurons can be fixed at a tip portion of a hollow tube or not. As shown in FIG. 11, it was confirmed that the micro bead was completely fixed. Thus, it has become possible to fix a microscopic target such as neurons. When the hollow tube of the hollow microtube structure used in this experiment was constituted such that a tip portion of an electrically insulating coating film layer projected from a tip portion of a metal coating film layer, measurement showed that a joint portion between the micro bead and the metal coating film layer had a seal resistance of several giga ohms. This fact indicates that the micro bead was not in contact with the metal coating film layer.

### REFERENCE SIGNS LIST

2 a substrate

4 a hollow tube

6 a metal coating film layer

8 an electrically insulating coating film layer

10 a light-emitting diode

12 chemical fluid

14 a syringe

16 an amp filter

18 light

20 a flexible tube

22 a metal film

24 a probe

26 a metal alloy film

28 a film

30 metal wire

32 normal saline solution

34 a connector

36 a micro bead used in a test

38 a microchannel-equipped resin

1-11. (canceled)

12. A hollow microtube structure, comprising:
a semiconductor substrate;
at least one hollow tube having a hollow portion extending rectilinearly in a perpendicular direction to a front surface of the semiconductor substrate, and provided in a hollow cylindrical shape on a micro scale on the front surface;
a metal coating film layer constituting an inner surface of the hollow tube;
an electrically insulating coating film layer constituting an outer surface of the hollow tube; and
a through hole extending in alignment, while communicated, with the hollow portion of the hollow tube and reaching a back surface of the semiconductor substrate.

13. The hollow microtube structure according to claim 12, wherein:
the semiconductor substrate is a silicon substrate,
the hollow tube has a two-layer structure comprising the metal coating film layer and the electrically insulating coating film layer, and
a layered body comprising a metal coating film layer and an electrically insulating coating film layer, continuing from the respective layers constituting the hollow tube, is laid on the front surface of the silicon substrate with the electrically insulating coating film layer disposed on an outer side.

14. The hollow microtube structure according to claim 12, wherein the at least one hollow tube is a plurality of hollow tubes formed in an array on the front surface of the substrate.

15. The hollow microtube structure according to claim 13, wherein the at least one hollow tube is a plurality of hollow tubes formed in an array on the front surface of the substrate.

16. A biopsy device using the hollow microtube structure according to claim 12, and comprising:
the hollow microtube structure;
chemical fluid injecting means provided continuously to an opening portion of the through hole on a back side of the substrate and supplying chemical fluid to an interior of the hollow portion of one of the at least one hollow tube; and
electric signal transmitting means and electrical measuring means each electrically connected to the one or another of the at least one hollow tube.

17. A biopsy device using the hollow microtube structure according to claim 12, and comprising:
the hollow microtube structure;
optical signal generating means for emitting light to enter from an opening portion of the through hole on a back side of the substrate and pass through one of the at least one hollow tube; and
optical measuring means for receiving reflected light which has reached the back side of the substrate through an interior of the hollow portion of another of the at least one hollow tube.

18. A biopsy device using the hollow microtube structure according to claim 12, and comprising:
the hollow microtube structure;
optical signal generating means for emitting light to enter from an opening portion of the through hole on a back side of the substrate and pass through one of the at least one hollow tube; and
electrical measuring means electrically connected to the one or another of the at least one hollow tube.

19. The biopsy device according to claim 17, further comprising chemical fluid injecting means provided continuously to the opening portion of the through hole on the back side of the substrate and supplying chemical fluid through an interior of the one or another of the at least one hollow tube.

20. The biopsy device according to claim 18, further comprising chemical fluid injecting means provided continuously to the opening portion of the through hole on the back side of the substrate and supplying chemical fluid through an interior of the one or another of the at least one hollow tube.

21. The biopsy device according to claim 16, further comprising:
fluid extracting means provided continuously to the opening portion of the through hole on the back side of the substrate and extracting, by suction, body fluid or chemical fluid by way of an interior of the one or another of the at least one hollow tube; and
chemical measuring means provided continuously to or on a way to the fluid extracting means.

22. The biopsy device according to claim 17, further comprising:
fluid extracting means provided continuously to the opening portion of the through hole on the back side of the substrate and extracting, by suction, body fluid or chemical fluid by way of an interior of the one or another of the at least one hollow tube; and
chemical measuring means provided continuously to or on a way to the fluid extracting means.

23. The biopsy device according to claim 18, further comprising:
fluid extracting means provided continuously to the opening portion of the through hole on the back side of the substrate and extracting, by suction, body fluid or chemical fluid by way of an interior of the one or another of the at least one hollow tube; and
chemical measuring means provided continuously to or on a way to the fluid extracting means.

24. A method for producing a hollow microtube structure, comprising:
an etching step of etching both a front surface and a back surface of a semiconductor substrate;
a sacrificial layer forming step of forming a cylindrical body in an etched area of a front side of the semiconductor substrate;
a metal coating film layer forming step of forming a metal coating film layer around the cylindrical body;
an electrically insulating coating film layer forming step of forming, around the metal coating film layer, an electrically insulating coating film layer comprising a different material from the cylindrical body;
a tip portion removing step of removing the electrically insulating coating film layer and the metal coating film layer of a tip portion of the cylindrical body, thereby exposing the tip portion of the cylindrical body; and
a piercing step of removing the cylindrical body and piercing the semiconductor substrate.

25. The method for producing a hollow microtube structure according to claim 24, wherein:
the etching step includes a step of forming a film on both the front surface and the back surface of the semiconductor substrate, a step of removing part of the films, and a step of etching film-removed regions of the semiconductor substrate; and
the piecing step is a step of removing the cylindrical body and piecing the semiconductor substrate until reaching one of the etched areas of the semiconductor substrate.

26. The method for producing the hollow microtube structure according to claim 24, wherein:
the metal coating film layer forming step is a step of forming a metal coating film layer on the front surface of the substrate simultaneously with forming the metal coating film layer around the cylindrical body, and
the electrically insulating coating film layer forming step is a step of forming an electrically insulating coating film layer on the metal coating film layer disposed on the front surface of the substrate simultaneously with forming the electrically insulating coating film layer around the metal coating film layer disposed around the cylindrical body.

27. The method for producing the hollow microtube structure according to claim 25, wherein:
the metal coating film layer forming step is a step of forming a metal coating film layer on the front surface of the substrate simultaneously with forming the metal coating film layer around the cylindrical body, and
the electrically insulating coating film layer forming step is a step of forming an electrically insulating coating film layer on the metal coating film layer disposed on the front surface of the substrate simultaneously with forming the electrically insulating coating film layer around the metal coating film layer disposed around the cylindrical body.

28. The biopsy device according to claim 19, further comprising:
fluid extracting means provided continuously to the opening portion of the through hole on the back side of the substrate and extracting, by suction, body fluid or chemical fluid by way of an interior of the one or another of the at least one hollow tube; and
chemical measuring means provided continuously to or on a way to the fluid extracting means.

29. The biopsy device according to claim 20, further comprising:
fluid extracting means provided continuously to the opening portion of the through hole on the back side of the substrate and extracting, by suction, body fluid or chemical fluid by way of an interior of the one or another of the at least one hollow tube; and
chemical measuring means provided continuously to or on a way to the fluid extracting means.
